(19) United States
(12) Patent Application Publication
(10) Pub. No.: US 2026/0096826 A1
(43) Pub. Date: Apr. 9, 2026

(54) DEVICES AND METHODS FOR ANATOMICAL OBSTRUCTION EXTRACTION

(71) Applicants: Boston Scientific Medical Device Limited, Galway (IE); Boston Scientific Scimed, Inc., Maple Grove, MN (US)

(72) Inventors: Deepak Kumar Sharma, Gurgaon (IN); Andrew James Schaubhut, Bolton, MA (US); Sharath Kumar G, Kanakapura (IN); Nachiket Gole, Pune (IN); Rajivkumar Singh, Pune (IN); James J. Scutti, Norwell, MA (US)

(73) Assignees: Boston Scientific Medical Device Limited, Galway (IE); Boston Scientific Scimed, Inc., Maple Grove, MN (US)

(21) Appl. No.: 19/348,042

(22) Filed: Oct. 2, 2025

Related U.S. Application Data
(60) Provisional application No. 63/702,904, filed on Oct. 3, 2024.

(51) Int. Cl. A61B 17/221; A61B 17/22

(52) U.S. Cl. CPC A61B 17/221; A61B 2017/22038; A61B 2017/2212

(57) ABSTRACT

Anatomical obstruction extraction devices and associated methods are disclosed herein. Example anatomical obstruction extraction devices may include a first elongate shaft including a first expandable portion and a first proximal portion extending in a proximal direction from the first expandable portion. The first expandable portion may include one or more elongate support elements. A first actuator may be provided and adapted to actuate the first expandable portion to adjust between an elongate configuration and an expanded configuration. A second elongate shaft may be disposed concentrically about the first elongate shaft, and may include a second expandable portion connected and/or otherwise coupled to one or more second actuators. The one or more second actuators may act upon and/or actuate the second expandable portion to adjust between an elongate configuration and an expanded configuration.

### CROSS-REFERENCE TO RELATED APPLICATIONS

This application claims the benefit of U.S. Provisional Patent Application Ser. No. 63/702,904 filed on Oct. 3, 2024, the disclosure of which is incorporated herein by reference.

### TECHNICAL FIELD

The present disclosure is directed to medical devices and associated methods. More particularly, the present disclosure relates to anatomical obstruction extraction devices and associated methods that may be utilized for treatment within various anatomical systems and/or subsystems of a subject, including but not limited to the gastrointestinal (GI) tract, the biliary tract, the vasculature, and/or the like.

### BACKGROUND

Anatomical obstructions such as biliary stones, kidney stones, calculi, thrombi, emboli and the like present challenging difficulties for effective and safe removal from body lumens. In some examples, a physician and/or practitioner may find an obstruction within one or more various anatomical systems within a subject that may be greater than 15 mm in its greatest dimension (i.e., width, length, diameter, height, etc.) Obstructions of this magnitude present inherent complications of removal given their size, and the stress these large obstructions may place on the interior luminal walls of the subject.

Additional complications may arise during the attempted removal of an obstruction as described above. In many procedures, a ballooned catheter is inserted proximate and/or across and/or along a desired area of treatment (e.g., biliary tract, gastrointestinal tract, blood vessel, or the like). After placement within the desired treatment area, one or more balloons of the ballooned catheter may be inflated, however, within the scenario of a removal of a large obstruction, balloon burst may be common and present further deleterious outcomes for the subject undergoing such procedures.

### BRIEF SUMMARY

This disclosure provides design, material, method, and use alternatives for medical devices, including but not limited to anatomical obstruction extraction devices.

An example medical device of the present disclosure may include a first elongate shaft comprising a first expandable portion and a first proximal portion extending in a proximal direction from the first expandable portion, the first expandable portion comprising one or more elongate support elements, a first actuator configured to actuate the first expandable portion between an elongate configuration and an expanded configuration, a second elongate shaft concentrically disposed about the first elongate shaft, the second elongate shaft comprising a second expandable portion and a second proximal portion extending in a proximal direction from the second expandable portion, the second expandable portion comprising one or more elongate support elements, and one or more second actuators configured to actuate the second expandable portion between an elongate configuration and an expanded configuration.

Alternatively or additionally to any of the examples disclosed herein, one or both of the first expandable portion and the second expandable portion may include a flexible covering.

Alternatively or additionally to any of the examples disclosed herein, the flexible covering may be one or more of a balloon, a stent, a sleeve, and a basket.

Alternatively or additionally to any of the examples disclosed herein, the flexible covering may be substantially porous.

Alternatively or additionally to any of the examples disclosed herein, the first expandable portion may include a first balloon covering the one or more first elongate support elements and the first elongate shaft may include a first inflation port located at the first proximal portion, the first inflation port may be configured and/or otherwise adapted to be in fluid communication with the balloon and/or to receive inflation fluid.

Alternatively or additionally to any of the examples disclosed herein, the one or more first elongate support elements of the first expandable portion may comprise a plurality of circumferentially spaced elongate struts.

Alternatively or additionally to any of the examples disclosed herein, one or both of the one or more first elongate support elements of the first expandable portion and the one or more second elongate support elements of the second expandable portion may be longitudinally biased toward one of the first elongate configuration and the second elongate configuration.

Alternatively or additionally to any of the examples disclosed herein, the first actuator may comprise one or more first pull wires coupled with the first expandable portion and the second actuator may comprise one or more second pull wires coupled with the second expandable portion.

Alternatively or additionally to any of the examples disclosed herein, the first expandable portion may be longitudinally adjustable with the first proximal portion when in an expanded configuration and the second expandable portion maybe longitudinally adjustable with the second proximal portion when in an expanded configuration.

Alternatively or additionally to any of the examples disclosed herein, the first elongate shaft may include a central lumen.

In other non-limiting examples, the present disclosure provides an anatomical obstruction extraction device. An anatomical obstruction extraction device of the present disclosure may include a first elongate shaft. The first elongate shaft may include a first proximal portion and a first distal portion. a plurality of first elongate struts may extend between the first proximal portion and the first distal portion and may be circumferentially spaced from one another. A first flexible covering extending over the plurality of first elongate struts. One or more first actuators may be coupled with the first distal portion. The plurality of first elongate struts may be configured to longitudinally and radially adjust in response to relative longitudinal movement between the first proximal portion and the one or more first actuators. A second elongate shaft may be configured to be concentrically disposed about the first elongate shaft. The first elongate shaft and the second elongate shaft may be configured to be longitudinally adjustable with respect to one another. The second elongate shaft may include second proximal portion and a second distal portion, a plurality of second elongate struts extending between the second proximal portion and the second distal portion and may be circumferentially spaced from one another, a second flexible covering extending over the second plurality of elongate struts, and one or more second actuators coupled with the second distal portion. The plurality of second elongate struts may be configured and/or otherwise adapted to longitudinally and radially adjust in response to relative longitudinal movement between the second proximal portion and the one or more second actuators.

Alternatively or additionally to any of the examples disclosed herein, the plurality of first elongate struts and the plurality of second elongate struts may be biased in a longitudinal direction.

Alternatively or additionally to any of the examples disclosed herein, one or both of the first proximal portion and the second proximal portion may include an inflation port.

Alternatively or additionally to any of the examples disclosed herein, each of the first flexible covering and the second flexible covering may be one or more of a balloon, a stent, a sleeve, and a basket.

Alternatively or additionally to any of the examples disclosed herein, the first flexible covering and the plurality of first elongate struts are longitudinally adjustable with the first proximal portion and the second flexible covering and the plurality of second elongate struts are longitudinally adjustable with the second proximal portion.

Alternatively or additionally to any of the examples disclosed herein, one or both of the one or more first actuators and the one or more second actuators may comprise one or more pull wires.

Alternatively or additionally to any of the examples disclosed herein, the first elongate shaft may include a central lumen.

Methods are also contemplated by the present disclosure and may incorporate any structure, function and/or inherency of any elements and features described in the present disclosure, whether individually, collectively, and/or aggregately. Applicable to any of the examples disclosed herein, a method of removing an obstruction from a biliary tract of a subject may include inserting a guidewire through a dilated access region proximate the biliary tract, guiding an obstruction extraction device over the guidewire and positioning the obstruction extraction device proximate the obstruction within the biliary tract, advancing a first expandable portion of a first elongate shaft to a location in the biliary tract distal of the obstruction, the first expandable portion comprising a first elongate support structure and a first covering, expanding a second expandable portion of a second elongate shaft at a location in the biliary tract proximal of the obstruction, the second expandable portion comprising a second elongate support structure and a second covering, expanding the first expandable portion at the location distal of the obstruction, and retracting the first elongate shaft and the first expandable portion in a proximal direction toward the second expandable portion to advance the obstruction in the proximal direction.

Alternatively or additionally to any of the examples disclosed herein, methods may include contracting the second expandable portion, retracting the second expandable portion in the proximal direction after contracting the second expandable portion, and expanding the second expandable portion after retracting the second expandable portion.

Alternatively or additionally to any of the examples disclosed herein, methods may include wherein the covering of the first expandable portion includes one or more of a balloon, a stent, a sleeve, and a basket.

Alternatively or additionally to any of the examples disclosed herein, methods may include wherein the elongate support structure of the first expandable portion includes a plurality of elongate struts circumferentially spaced from one another.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE SEVERAL VIEWS OF THE DRAWINGS

The drawings illustrate the design and utility of configurations of the present disclosure. It should be noted that the figures are not drawn to scale and that elements of similar structures or functions are represented by like reference numerals throughout the figures. The drawings are not to be considered as limiting the scope of the disclosure. The present disclosure is described and explained with additional specificity and detail through the use of the accompanying drawings, in which:

FIG. 1 illustrates a schematic view of an example of accessing the biliary duct of a subject with a medical device system;

FIG. 2 illustrates a schematic view of an obstruction lodged within the biliary tract of a subject;

FIG. 3 illustrates a schematic view of a portion of an example configuration of a medical device;

FIG. 4 illustrates a schematic view of the portion of the example configuration of the medical device depicted in FIG. 3, in a first position;

FIG. 5 illustrates a schematic detailed view from the magnification (5) in FIG. 4;

FIG. 6 illustrates a schematic view of the portion of the example configuration of the medical device depicted in FIG. 3, in a second position;

FIG. 7 illustrates a schematic view of a portion of an example configuration of a medical device;

FIG. 8A illustrates a schematic view of a portion of an example configuration of a medical device;

FIG. 8B illustrates a schematic detailed view from the magnification (8B) in FIG. 8A;

FIG. 8C illustrates a schematic detailed view from the magnification (8C) in FIG. 8A;

FIG. 9 illustrates a schematic view of a portion of an example configuration of a medical device, in a first position;

FIG. 10 illustrates a schematic view of the portion of the medical device depicted in FIG. 9, in a second position; and

FIGS. 11A-11E illustrate schematic views of a portion of an example medical device being used in a method of removing an obstruction from a body lumen.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

The presence of an anatomical obstruction in and/or along the biliary tract can lead to serious conditions, such as, but not limited to, obstructive jaundice, acute suppurative cholangitis, sepsis, and/or gallstone pancreatitis. Prior to the implementation of endoscopic retrograde cholangiopancreatography (ERCP), extraction of an anatomical obstruction (e.g., biliary stone, biliary calculi) required procedures such as laparotomy and open common bile duct exploration, a surgery that was associated with significant mortality, morbidity, and/or prolonged inpatient convalescence. With the advent of ERCP, anatomical obstruction extraction can be accomplished by a gastroenterologist or other qualified practitioners as an outpatient procedure with minimal risks.

The ERCP procedure involves the introduction of a side-viewing endoscope such as a duodenoscope through the mouth of a subject and advancement into the second portion of the duodenum, with the subject positioned semi-prone on an X-ray table for fluoroscopic imaging. The tip of the duodenoscope is aligned with the bile duct and pancreatic duct openings at a shared protuberance called the ampulla or papilla of Vater. The papilla of Vater has an opening called the ampullary orifice surrounded by a sphincter muscle called the sphincter of Oddi. Thereafter, imaging may be performed to provide an assessment of the biliary tract and associated anatomical structures. If a blockage or other anatomical obstruction is detected or found, a device may be inserted through a lumen of the endoscope and/or inserted adjacent or proximate to an endoscope for the purposes of removing the anatomical obstruction from the biliary tract and associated anatomical structures. To address many unmet needs in the state-of-the-art, the present disclosure provides anatomical obstruction extraction devices and associated methods which effectively remove, transport, and/or clear anatomical obstructions from various body lumens and/or cavities of a subject.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

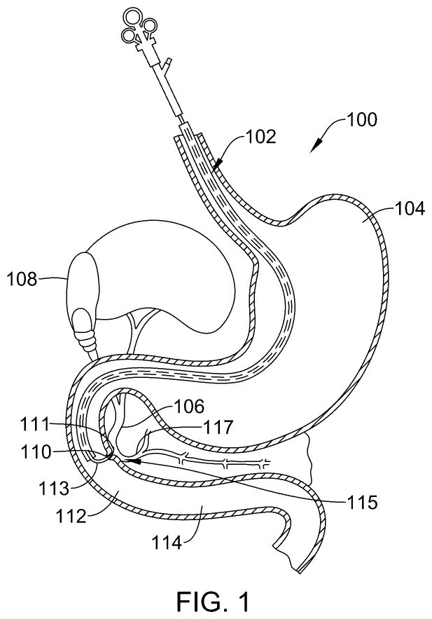

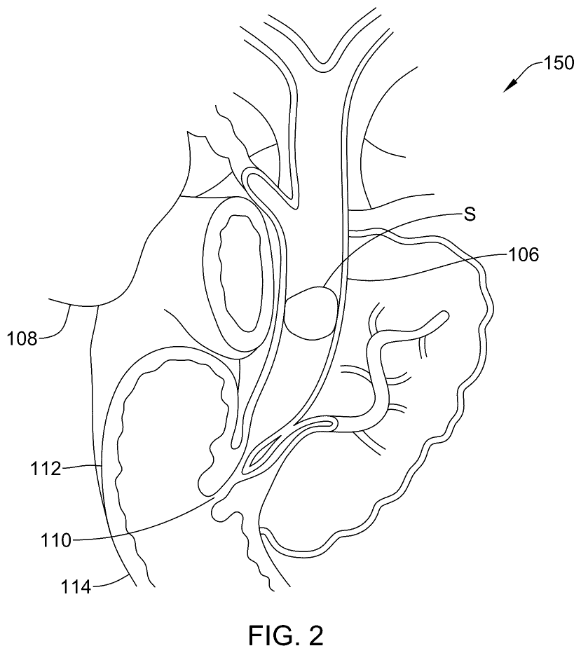

FIG. 1 depicts a schematic view of an example procedural site 100 (such as a biliary and digestive tract) of a subject where an endoscope 102 (e.g., a duodenoscope, etc.) or like introducer bearing an accessory medical device 113 has been inserted into the gastrointestinal tract, through the stomach 104, into the small intestine 114, and into the papilla of Vater 110 by way of the duodenum 112. The biliary duct 106 leads to the gallbladder 108, and forms part of the biliary tract in which anatomical obstructions can form and further accrete into large anatomical obstructions, such as an anatomical obstruction S (e.g., a stone and/or other suitable anatomical structure including, but not limited to, a biliary calculi, mass, osteoid, clot, tissue deformity, tissue anomaly, luminal deformity, luminal anomaly) shown in FIG. 2.

More specifically, FIG. 1 depicts a schematic view of an illustrative selective cannulation during an ERCP procedure, which includes a guidewire 111 and/or the accessory medical device 113 (e.g., an endoscopic accessory device, such as a sphincterotome, stone removable device, and/or other suitable accessory medical device) being passed towards, against, and/or through a body lumen such as the papilla of Vater 110 (e.g., ampullary entry) near the duodenum 112 to access the Sphincter of Oddi Complex 115. During the cannulation procedure, a distal portion of the endoscope 102 may be positioned within the duodenum 112. The guidewire 111 and the accessory medical device 113 may be advanced through a working channel (e.g., a lumen) of the endoscope 102 towards the papilla of Vater 110. Additionally, the guidewire 11 and/or the accessory medical device 113 may be advanced against and/or through the papilla of Vater 110 to one of the biliary duct 106 and the pancreatic duct 117.

FIG. 2 depicts a schematic magnified view of a biliary tract of a subject (e.g., a patient). As depicted, an anatomical obstruction S may be present within the biliary duct 106 and require treatment by the medical devices disclosed herein. As shown in FIG. 2, the papilla of Vater 110 provides an access point to the biliary duct 106 from the duodenum 112 of the small intestine 114 such that the medical devices disclosed herein may achieve access to the anatomical obstruction S and remove the anatomical obstruction S from the biliary duct 106 as will be further described herein.

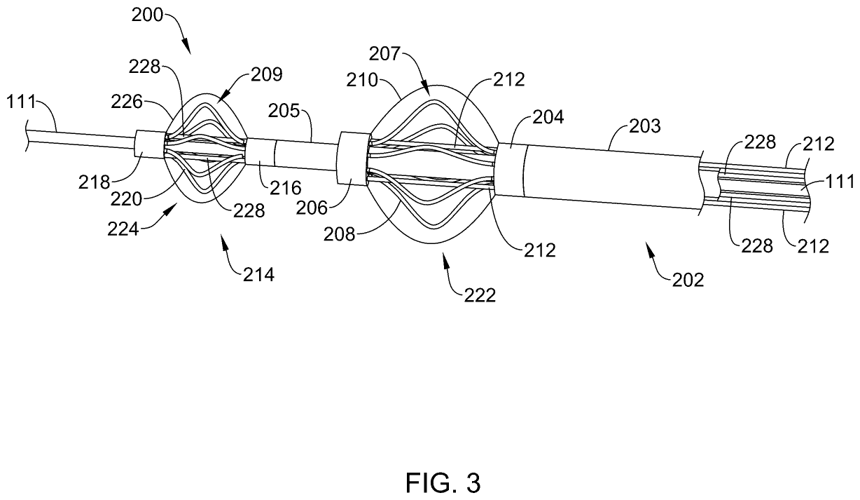

FIG. 3 depicts a distal end of an illustrative configuration of an extraction device 200 (e.g., an anatomical obstruction extraction device). The extraction device 200 may include a first elongate shaft 202 and a second elongate shaft 214, which may be connected and/or otherwise coupled to a handle or other like device (not shown) at and/or proximate its proximal end. In some examples, the second elongate shaft 214 may extend through a lumen (e.g., a central lumen or other suitable lumen) of the first elongate shaft 202 such that the first elongate shaft 202 is positioned about the second elongate shaft 214. In some examples, the first elongate shaft 202 and the second elongate shaft 214 may be configured to longitudinally adjust and/or translate with respect to one another. In some examples, the second elongate shaft 214 may have a length that is longer than a length of the first elongate shaft 202 to facilitate the second elongate shaft 214 extending beyond a distal end and/or a proximal end of the first elongate shaft 202. Proximal sections of the first elongate shaft 202 and the second elongate shaft 214 have been removed from the extraction device 200 depicted in FIG. 3 to depict components of the extraction device 200 extending through proximal portions of the first elongate shaft 202 and the second elongate shaft 214.

The first elongate shaft 202 may include a first proximal portion 203, a first expandable portion 222, and a first distal portion 206, where a proximal end of the first expandable portion 222 may be coupled with the first proximal portion 203 and a distal end of the first expandable portion 222 may be coupled with the first distal portion 206. In some examples, the first proximal portion 203 may extend in a proximal direction from the first expandable portion 222 and/or may include a first coupling component 204 at and/or proximate the distal end of the first proximal portion 203 such that the first proximal portion 203 may longitudinally translate and/or adjust with the first expandable portion 222, but other suitable configurations are contemplated. In some examples, the first expandable portion 222 may be part of and/or may be coupled with the first coupling component 204.

The first elongate shaft 202 may have any suitable structure. For example, the first elongate shaft 202 may be formed with or from a metal material, a polymer material, combinations of metal materials and polymer materials, one or more rods, one or more tubes, one or more hypotubes, one or more concentric tubes, one or more rings, one or more struts, one or more wires, one or more stents, one or more braids, one or more coverings, and/or materials or structures. In one example, the first elongate shaft 202 may include the first proximal portion 203 formed from a polymer tube, the first expandable portion 222 formed from one or more struts and a covering, and the first distal portion 206 formed form a ring. Other suitable configurations are contemplated.

The first coupling component 204 may have any suitable configuration. For example, the first coupling component 204 may be formed from a metal material, a polymer material, combinations of metal materials and polymer materials, a rigid component, a flexible component, a tube, a shaft, a ring, an annular portion, an annulus, a ring structure, an annular structure, and/or other suitable materials or structures. In one example, the first coupling component 204 may have a ring configuration.

The first distal portion 206 may have any suitable configuration. For example, the first distal portion 206 may be formed from a metal material, a polymer material, combinations of metal materials and polymer materials, a coupling component, a rigid component, a flexible component, a tube, a shaft, a ring, an annular portion, an annulus, a ring structure, an annular structure, and/or other suitable materials or structures. In one example, the first distal portion 206 may be a distal coupling component configured to couple with the first expandable portion 222 and may define a lumen. In one example, the first distal portion 206 may have a ring configuration.

The first expandable portion 222 may have any suitable configuration. In some examples, the first expandable portion 222 may include a first expandable support structure 207 (e.g., one or more elongate support elements and/or other suitable expandable support structure) and/or one or more first flexible coverings 210. In one example, the first flexible covering 210 may extend over an entirety of or at least a portion of the first expandable support structure 207. In some examples, the first flexible covering 210 may be omitted.

The first flexible covering 210 may have any suitable configuration for covering the first expandable support structure 207 of the first expandable portion 222. In some examples, the first flexible covering 210 may be an expansible member, a flexible member, a resilient member, a porous member, a non-porous member, a balloon, a stent, a basket, a sleeve, a filter, and/or other suitable flexible covering. In one example, the first flexible covering 210 may be a porous member. In one example, the first flexible covering 210 may be a balloon. Although other configurations are contemplated, when the first flexible covering 210 is a balloon, the first elongate shaft 202 may include and/or define one or more lumens for receiving and passing inflation fluid (e.g., air, saline, inflation fluid, and/or other suitable fluid) to and/or from the balloon.

The first flexible covering 210 may be formed from any suitable material. For example, the first flexible covering 210 may be formed from a metal, a polymer, lubricious materials, flexible materials, resilient materials, silicone, and/or other suitable materials. In one example, the first flexible covering 210 and/or other flexible coverings disclosed herein may be composed of and/or include silicone. In some examples, the first flexible covering 210 may include or have thereon a coating at least partially and/or substantially encapsulating the first flexible covering 210. In this and other examples, the coating may be and/or may include silicone and/or other suitable lubricious materials.

The first expandable support structure 207 may have any suitable configuration configured to support the first flexible covering 210. For example, the first expandable support structure 207 may have one or more struts, one or more wires, one or more pull wires, one or more wires of varying cross-section, one or more wires of consistent cross-section, one or more rods, one or more shafts, one or more tubes, one or more posts, one or more hollow elements, one or more flexible elements, one or more substantially hollow elements, one or more non-hollow elements, one or more stent structures, and/or other suitable configurations. In one example, the first expandable support structure 207 may comprise a plurality of first elongate struts 208, as depicted for example in FIG. 3. In one example, the first expandable support structure 207 may include a stent structure.

The first elongate struts 208 and/or other suitable first expandable support structure 207 may be formed of any suitable materials. Example suitable materials include, but not limited to, nitinol, metals, alloys, polymers, thermoplastics, synthetic materials, flexible materials, resilient materials, shape-memory materials, and/or other suitable materials and/or any combination thereof.

When the first expandable support structure 207 includes the first elongate struts 208, the first elongate struts 208 may be positioned and/or situated in an array defining, surrounding, and/or proximate to one or more lumens extending through the extraction device 200. The one or more lumens may be centrally positioned within the interior diameter of the extraction device 200 and longitudinally coaxial and/or concentric with the extraction device 200. In other words, the one or more lumens may run along the interior radial center of the extraction device 200 in a coaxial relationship with concentrically disposed components of the extraction device 200 as will be described further herein. Other suitable lumen configurations are contemplated.

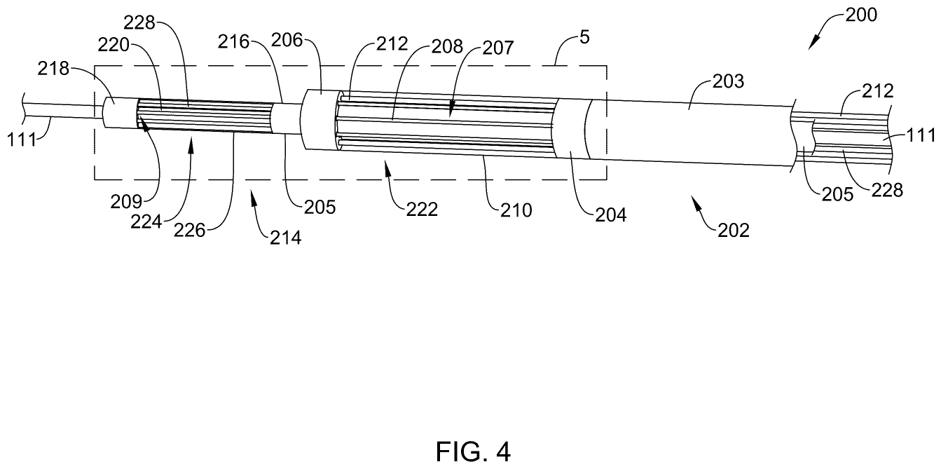

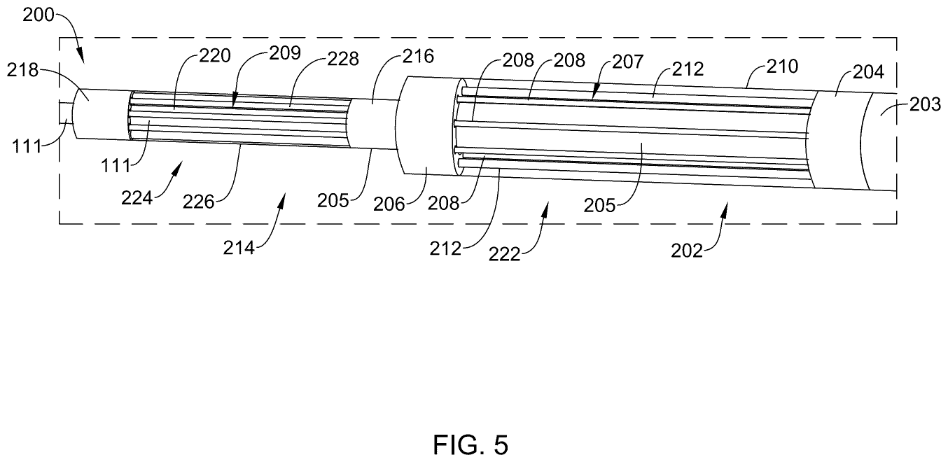

The first expandable support structure 207 (e.g., the first elongate struts 208, etc.) may be adapted and/or otherwise configured to expand radially and/or laterally. For example, the first expandable support structure 207 may be coupled with one or more first actuators 212 and upon application of force to and/or actuation of the one or more first actuators 212, the first expandable support structure 207 and thus the first expandable portion 222 may adjust between a first position (e.g., a collapsed or elongate configuration) in which the first expandable support structure 207 may be longitudinally extended and radially compressed (e.g., as depicted in FIG. 4) and a second position (e.g., an expanded configuration) in which the first expandable support structure 207 may be longitudinally compressed and radially expanded (e.g., as depicted in FIGS. 3 and 5). In one example, actuation of the one or more first actuators 212 a first time (e.g., in a first direction, such as a proximal direction) may cause the first expandable support structure 207 and/or the first expandable portion 222 to adjust from the first position to the second position and when the one or more first actuators 212 are actuated a second time (e.g., in a second direction, such as a distal direction), the first elongate struts 208 and/or the first expandable portion 222 may adjust from the second position to the first position.

One or more of the first elongate struts 208 and/or other suitable component(s) of the first expandable support structure 207 may be biased to one of the first position and the second position. In one example, actuation of the one or more first actuators 212 may cause the first expandable support structure 207 and/or the first expandable portion 222 to adjust from the first position to the second position when the first elongate struts 208 are biased to the first position. In one example, actuation of the one or more first actuators 212 may cause the first expandable support structure 207 and/or the first expandable portion 222 to adjust from the second position to the first position when the first elongate struts 208 are biased to the second position. When the one or more first actuators 212 are released or unactuated, a bias force of the first expandable support structure 207 may cause the first expandable support structure 207 to return to the position in which it is biased towards.

The first actuators 212 may have any suitable configuration. For example, the first actuators 212 may be or may include pull wires, tension wires, actuation shafts, actuation rods, strings, tubes, lumens, fluid, buttons, slides, wheels, and/or other suitable actuators or actuation components. The first actuators 212 may be provided in an array, a series, a circular or circumferential array, a substantially circular array, an ovular array, a substantially ovular array, an elliptical array, a substantially elliptical array, and/or any array that encapsulates and/or otherwise surrounds and/or partially surrounds a lumen through which the second elongate shaft 214 may extend. In one example configuration of the first actuators 212, the first actuators 212 may include one or more pull wires having a distal end coupled with the first distal portion 206, as depicted for example in FIG. 3, and/or otherwise coupled with the first expandable portion 222. In one example configuration of the first actuators 212, the first actuators 212 may include one or more elongate tubes coupled with the first distal portion 206 and/or otherwise coupled with the first expandable portion 222, where the elongate tube may or may not be concentric with the first proximal portion 203.

The first actuators 212 may act upon the first expandable support structure 207 in any suitable manner. In some examples, when the first actuators 212 include one or more pull wires coupled with the first distal portion 206, a force in a proximal direction may be applied to the pull wires to withdraw the first distal portion 206 relative to the first coupling component 204 and laterally and/or radially expand the plurality of first elongate struts 208, as depicted for example in FIG. 3, such that the plurality of first elongate struts 208 may protrude beyond initial lateral and/or radial boundaries of the extraction device 200. In other words, the first actuators 212 may act upon the plurality of first elongate struts 208 such that the first expandable portion 222 may expand beyond an outer circumference of one or both of the first proximal portion 203 and the first distal portion 206.

The second elongate shaft 214 may include a second proximal portion 205, a second expandable portion 224, and a second distal portion 218, where a proximal end of the second expandable portion 224 may be coupled with the second proximal portion 205 and a distal end of the second expandable portion 224 may be coupled with the second distal portion 218. In some examples, the second proximal portion 205 may extend in a proximal direction from the second expandable portion 224 and/or may include a second coupling component 216 at and/or proximate the distal end of the second proximal portion 205 such that the second proximal portion 205 may longitudinally translate and/or adjust with the second expandable portion 224, but other suitable configurations are contemplated. In some examples, the second expandable portion 224 may be part of and/or may be coupled with the second coupling component 216.

The second elongate shaft 214 may have any suitable structure. For example, the second elongate shaft 214 may be formed from a metal material, a polymer material, combinations of metal materials and polymer materials, one or more rods, one or more tubes, one or more hypotubes, one or more concentric tubes, one or more rings, one or more struts, one or more wires, one or more stents, one or more braids, one or more coverings, and/or materials or structures. In one example, the second elongate shaft 214 may include the second proximal portion 205 formed from a polymer tube, the second expandable portion 224 formed from one or more struts and a covering, and the second distal portion 218 formed form a ring.

The second coupling component 216 may have any suitable configuration. For example, the second coupling component 216 may be formed from a metal material, a polymer material, combinations of metal materials and polymer materials, a rigid component, a flexible component, a tube, a shaft, a ring, an annular portion, an annulus, a ring structure, an annular structure, and/or other suitable materials or structures. In one example, the second coupling component 216 may have a ring configuration.

The second distal portion 218 may have any suitable configuration. For example, the second distal portion 218 may be formed from a metal material, a polymer material, combinations of metal materials and polymer materials, a coupling component, a rigid component, a flexible component, a tube, a shaft, a ring, an annular portion, an annulus, a ring structure, an annular structure, and/or other suitable materials or structures. In one example, the second distal portion 218 may be a distal coupling component configured to couple with the second expandable portion 224 and may define a lumen.

The second expandable portion 224 may have any suitable configuration. In some examples, the second expandable portion 224 may include a second expandable support structure 209 (e.g., one or more elongate support elements and/or other suitable expandable support structure) and/or one or more second flexible coverings 226. In one example, the second flexible covering 226 may extend over an entirety of or at least a portion of the second expandable support structure 209. In some examples, the second flexible covering 226 may be omitted.

The second flexible covering 226 may have any suitable configuration for covering the second expandable support structure 209 of the second expandable portion 224. In some examples, the second flexible covering 226 may be an expansible member, a flexible member, a resilient member, a porous member, a non-porous member, a balloon, a stent, a basket, a sleeve, a filter, and/or other suitable flexible covering. In one example, the second flexible covering 226 may be a balloon, but other suitable configurations are contemplated. In one example, the second flexible covering 226 may be a porous member. Although other configurations are contemplated, when the second flexible covering 226 is a balloon, the second elongate shaft 214 may include and/or define one or more lumens for receiving and passing inflation fluid to and/or from the balloon.

The second flexible covering 226 may be formed from any suitable material. For example, the second flexible covering 226 may be formed from a metal, a polymer, a lubricious materials, silicone, and/or other suitable materials. In one example, the second flexible covering 226 and/or other flexible coverings disclosed herein may be composed of and/or include silicone. In some examples, the second flexible covering 226 may include or have thereon a coating at least partially and/or substantially encapsulating the second flexible covering 226. In this and other examples, the coating may be and/or may include silicone and/or other suitable lubricious materials.

The second expandable support structure 209 may have any suitable configuration configured to support the second flexible covering 226. For example, the second expandable support structure 209 may have one or more struts, one or more wires, one or more pull wires, one or more wires of varying cross-section, one or more wires of consistent cross-section, one or more rods, one or more shafts, one or more tubes, one or more posts, one or more hollow elements, one or more flexible elements, one or more substantially hollow elements, one or more non-hollow elements, and/or other suitable configurations and/or any combinations thereof. In one example, the second expandable support structure 209 may comprise a plurality of second elongate struts 220, as depicted for example in FIG. 3.

The second elongate struts 220 and/or other suitable second expandable support structure 209 may be formed of any suitable materials. Example suitable materials include, but not limited to, nitinol, metals, alloys, polymers, thermoplastics, synthetic materials, flexible materials, resilient materials, shape-memory materials, and/or other suitable materials and/or any combination thereof.

When the second expandable support structure 209 includes the second elongate struts 220, the second elongate struts 220 may be positioned and/or situated in an array defining, surrounding, and/or proximate to one or more lumens extending through the extraction device 200. The one or more lumens may be centrally positioned within the interior diameter of the extraction device 200 and longitudinally coaxial and/or concentric with the extraction device 200. In other words, the one or more lumens may run along the interior radial center of the extraction device 200 in a coaxial relationship with concentrically disposed components of the extraction device 200 as will be described further herein. In one example, a lumen at least partially defined by the second elongate struts 220 may be configured to receive the guidewire 111. Other suitable lumen configurations are contemplated.

The second expandable support structure 209 (e.g., the second elongate struts 220, etc.) may be adapted and/or otherwise configured to expand radially and/or laterally. For example, the second expandable support structure 209 may be coupled with one or more second actuators 228 and upon application of force to and/or actuation of the one or more second actuators 228, the second expandable support structure 209 and thus the second expandable portion 224 may adjust between a first position (e.g., a collapsed or elongate configuration) in which the second expandable support structure 209 may be longitudinally extended and radially compressed (e.g., as depicted in FIG. 4) and a second position (e.g., an expanded configuration) in which the second expandable support structure 209 may be longitudinally compressed and radially expanded (e.g., as depicted in FIGS. 3 and 5). In one example, actuation of the one or more second actuators 228 a first time (e.g., in a first direction, such as a proximal direction) may cause the second expandable support structure 209 and/or the second expandable portion 224 to adjust from the first position to the second position and when the one or more second actuators 228 are actuated a second time (e.g., in a second direction, such as a distal direction), the second elongate struts 220 and/or the second expandable portion 224 may adjust from the second position to the first position.

One or more of the second elongate struts 220 and/or other suitable component(s) of the second expandable support structure 209 may be biased to one of the first position and the second position. In one example, actuation of the one or more second actuators 228 may cause the second expandable support structure 209 and/or the second expandable portion 224 to adjust from the first position to the second position when the second elongate struts 220 are biased to the first position. In one example, actuation of the one or more second actuators 228 may cause the second expandable support structure 209 and/or the second expandable portion 224 to adjust from the second position to the first position when the second elongate struts 220 are biased to the second position. When the one or more second actuators 228 are released or unactuated, a bias force of the second expandable support structure 209 may cause the second expandable support structure 209 to return to the position in which it is biased towards.

The second actuators 228 may have any suitable configuration. For example, the second actuators 228 may be or may include pull wires, tension wires, actuation shafts, actuation rods, strings, tubes, lumens, fluid, buttons, slides, wheels, and/or other suitable actuators. The second actuators 228 may be provided in an array, a series, a circular or circumferential array, a substantially circular array, an ovular array, a substantially ovular array, an elliptical array, a substantially elliptical array, and/or any array that encapsulates and/or otherwise surrounds and/or partially surrounds a lumen through which the guidewire 111 may extend. In one example configuration of the second actuators 228, the second actuators 228 may include one or more pull wires having a distal end coupled with the second distal portion 218, as depicted for example in FIG. 3, and/or otherwise coupled with the second expandable portion 224. In one example configuration of the second actuators 228, the second actuators 228 may include one or more elongate tubes coupled with the second distal portion 218 and/or otherwise coupled with the second expandable portion 224, where the elongate tube may or may not be concentric with the second proximal portion 205.

The second actuators 228 may act upon the second expandable support structure 209 in any suitable manner. In some examples including when the second actuators 228 include one or more pull wires coupled with the second distal portion 218, a force in a proximal direction may be applied to the pull wires to withdraw the second distal portion 218 relative to the second coupling component 216 and laterally and/or radially expand the plurality of second elongate struts 220, as depicted for example in FIG. 3, such that the plurality of second elongate struts 220 may protrude beyond initial lateral and/or radial boundaries of the extraction device 200. In other words, second actuators 228 may act upon the plurality of second elongate struts 220 such that the second expandable portion 224 may expand beyond an outer circumference of one or both of the second proximal portion 205 and the second distal portion 220.

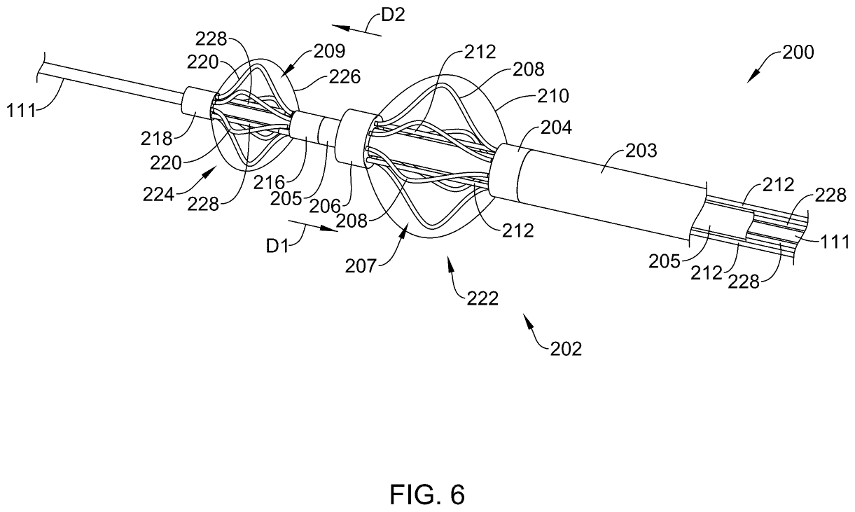

FIGS. 4-6 depict schematic views of the first expandable portion 222 and the second expandable portion 224 of the extraction device 200 depicted in FIG. 3 transitioning between the first position and the second position. FIG. 4 depicts a schematic view of the portion of the extraction device 200 depicted in FIG. 3, where the first expandable portion 222 and the second expandable portion 224 of the extraction device 200 are in the first position (e.g., a collapsed configuration) and the first elongate shaft 202 and the second elongate shaft 214 are longitudinally adjusted relative to one another such that more of the second proximal portion 205 is within the first elongate shaft 202 and the second expandable portion 224 is closer to the first expandable portion 222 than is depicted in FIG. 6. In some examples, the extraction device 200 may be delivered to a target area with one or both of the first expandable portion 222 and the second expandable portion 224 in the first position. In some examples, the first flexible covering 210 and/or the second flexible covering 226 may be clear such that contents of the first expandable portion 222 and the second expandable portion 224 may be viewable through the first flexible covering 210 and/or the second flexible covering 226, but other suitable configurations are contemplated and the first flexible covering 210 and/or the second flexible covering 226 may be opaque.

The first elongate shaft 202 and the second elongate shaft 214 may be longitudinally and/or rotationally adjusted with respect to one another in any suitable manner. In some examples, a handle coupled with the first elongate shaft 202 and/or the second elongate shaft 214 may be adjusted or actuated to longitudinally and/or rotationally adjust the first elongate shaft 202 and/or the second elongate shaft 214 relative to one another.

FIG. 5 shows a magnified or detailed view of selection 5 in FIG. 4. As depicted in FIG. 5, the guidewire 111 may extend through a lumen of the second elongate shaft 214 and the second elongate shaft 214 may extend through a lumen of the first elongate shaft 202. The plurality of first elongate struts 208 may have a proximal end coupled with the first coupling component 204 and the first distal portion 206. The pull wires of the first actuator 212 may be coupled with the first distal portion 206 and extend through and/or along the first coupling component 204 and/or the first proximal portion 203 of the first elongate shaft 202. The plurality of second elongate struts 220 may have a proximal end coupled with the second coupling component 216 and the second distal portion 218. The pull wires of the second actuator 228 may be coupled with the second distal portion 218 and extend through and/or along the second coupling component 216 and/or the second proximal portion 205 extending through the first elongate shaft 202.

The first elongate struts 208 and/or the pull wires of the first actuator 212 may be coupled with one or more components of the first elongate shaft 202 in any suitable manner. For example, the first elongate struts 208 and/or the pull wires of the first actuator 212 may be coupled with one or more components of the first elongate shaft 202 using adhesive, welding techniques, molding techniques, and/or other suitable coupling techniques.

The second elongate struts 220 and/or the pull wires of the second actuator 228 may be coupled with one or more components of the second elongate shaft 220 in any suitable manner. For example, the second elongate struts 220 and/or the pull wires of the second actuator 228 may be coupled with one or more components of the second elongate shaft 214 using adhesive, welding techniques, molding techniques, and/or other suitable coupling techniques.

FIG. 6 depicts a schematic view of the portion of the extraction device depicted in FIG. 3, where the first expandable portion 222 and the second expandable portion 224 of the extraction device 200 are in the second position (e.g., an expanded configuration) and the first elongate shaft 202 and the second elongate shaft 214 are longitudinally adjusted relative to one another such that less of the second proximal portion 205 is within the first elongate shaft 202 and the second expandable portion 224 is further distal from first expandable portion 222 than is depicted in FIG. 4. For example, the first elongate shaft 202 may be advanced in a first direction D1 (e.g., in a proximal direction) and/or the second elongate shaft 214 may be advanced in a second direction D2 (e.g., in a distal direction) to longitudinally adjust the first elongate shaft 202 and the second elongate shaft 214 relative to one another.

A user may radially adjust and/or longitudinally adjust the first expandable portion 222 and/or the second expandable portion 224 to facilitate dilating a body lumen and/or to facilitate moving one or more stones or other suitable obstructions within the body lumen. The first expandable portion 222 and/or the second expandable portion 224 may be radially and/or longitudinally adjusted with or independent of the other of the second expandable portion 224 and the first expandable portion 222. Although not depicted in FIG. 6, the first expandable portion 222 and the first elongate shaft 202 may be rotationally adjusted with or independent of the second expandable portion 224 and the second elongate shaft 214.

Movement of any components relative to any other components of the extraction device 200 disclosed herein may be carried out in a longitudinal direction (i.e., along the length of the devices disclosed herein in any direction) and may include translational movement (i.e., relative and/or slidable movement between components of devices in a proximal direction, distal direction, or any linear direction). Desired movement of a portion of one or both of the first elongate shaft 202 and second elongate shaft 214 relative to another portion of the first elongate shaft 202 and the second elongate shaft 21, respectively, may result in lateral and/or radial expansion of the first expandable portion 222 and the second expandable portion 224, respectively. Actuation of one or more of the first actuators 212 and second actuators 228 and/or any additional actuators contemplated herein may result in the lateral and/or radial expansion of the first expandable portion 222 and/or the second expandable portion 224.

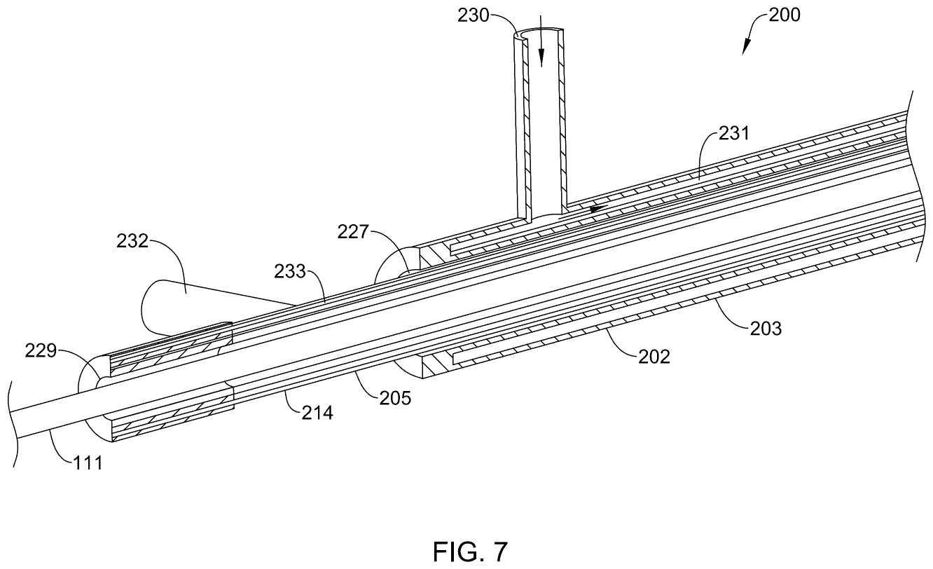

FIG. 7 depicts a schematic partial cross-sectional view of a portion of a configuration of the extraction device 200, where the first elongate shaft 202 and the second elongate shaft 214 are depicted in cross-section and the guidewire 111 is depicted from a side view. The configuration of the extraction device 200 depicted in FIG. 7 includes fluid ports for receiving inflation fluid for adjusting an outer diameter of the first expandable portion 222 and the second expandable portion 224.

As shown in FIG. 7, the second elongate shaft 214 may be nested and/or otherwise coaxially and/or slidably and/or telescopically disposed within the first elongate shaft 202. For example, the second elongate shaft 214 may extend through a first lumen 227 extending between a proximal end and a distal end of the first elongate shaft 202. In some examples, the second elongate shaft 214 may include a second lumen 229 extending between a proximal end and a distal end of the second elongate shaft 214, where the second lumen 229 is configured to receive the guidewire 111.

The first elongate shaft 202 may include a first inflation port 230 disposed anywhere within, along, about, and/or proximate the first elongate shaft 202 including, but not limited to, being disposed within a wall of the first elongate shaft 202. In one example, the first inflation port 230 may be located at or proximate a proximal end of the first proximal portion 203 of the first elongate shaft 202, but other suitable configurations are contemplated.

The first inflation port 230 may be in fluid communication with a first inflation lumen 231 and the first flexible covering 210 configured as a balloon. The first inflation lumen 231 may extend along the first elongate shaft 202 to the first expandable portion 222 (not shown in FIG. 7). In some examples, the first inflation lumen 231 may be a concentric lumen about the first lumen 227 and may extend through a wall of the first elongate shaft 202 (e.g., through at least a wall of the second proximal portion 205), as depicted for example in FIG. 7, but other suitable configurations are contemplated.

The second elongate shaft 214 may include a second inflation port 232 disposed anywhere within, along, about, and/or proximate the second elongate shaft 214 including, but not limited to, being disposed within a wall of the second elongate shaft 214. In one example, the second inflation port 232 may be located at or proximate a proximal end of the second proximal portion 205 of the second elongate shaft 214, but other suitable configurations are contemplated. As depicted in FIG. 7, the second inflation port 232 may be located in the second elongate shaft 214 such that the second inflation port 232 is proximal of the proximal end of the first proximal portion 203 of the first elongate shaft 202.

The second inflation port 232 may be in fluid communication with a second inflation lumen 233 (e.g., in a manner similar to how the first inflation port 230 is in fluid communication with the first inflation lumen 231 and/or in one or more other suitable manners) and the second flexible covering 226 configured as a balloon. The second inflation lumen 233 may extend along the second elongate shaft 214 to the second expandable portion 224 (not shown in FIG. 7). In some examples, the second inflation lumen 233 may be a concentric lumen about the second lumen 229 and may extend through a wall of the second elongate shaft 214 (e.g., through at least a wall of the second proximal portion 205), as depicted for example in FIG. 7, but other suitable configurations are contemplated.

The extraction devices 200 disclosed herein may include one or more inflation ports, two or more inflation ports, three or more inflation ports, etc. The inflation ports of any of the examples disclosed herein may take the form and/or be alternatively referred to as apertures, infusion ports, saline ports, expansion ports, and/or any other suitable type of port or opening or passage configured to receive fluid.

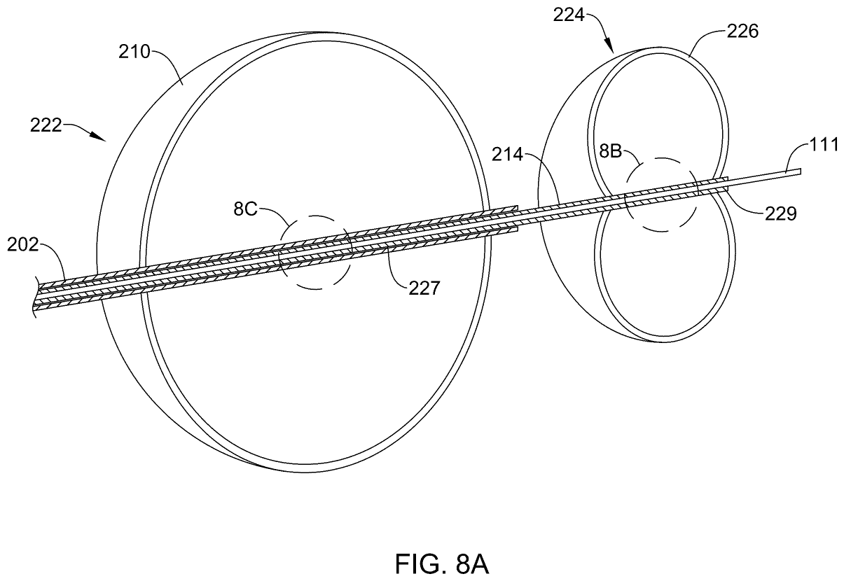

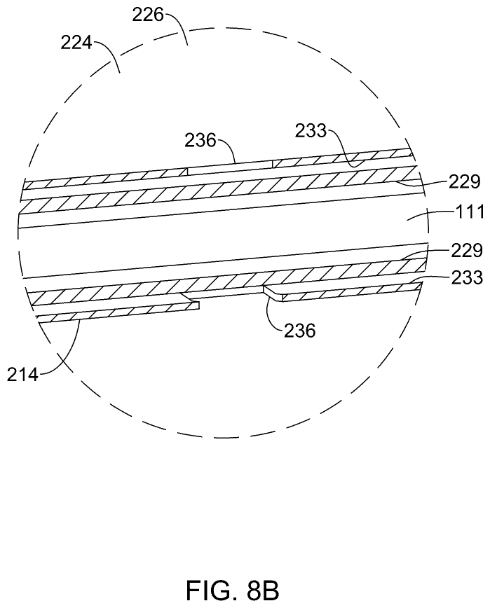

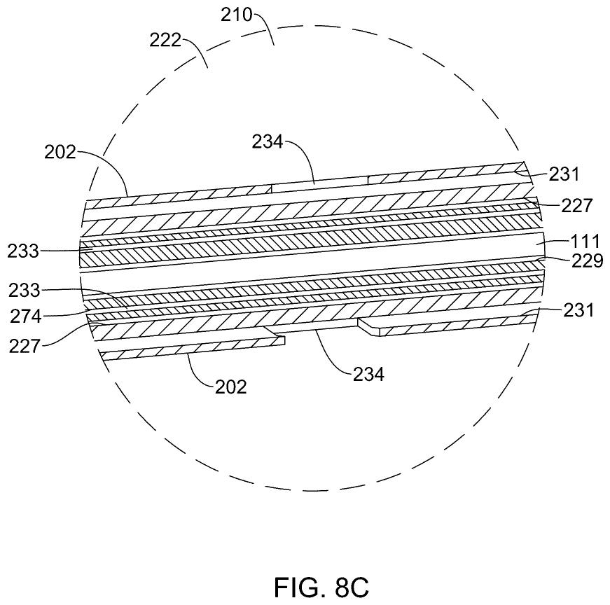

FIGS. 8A-8C depicts a schematic partial cross-section view of a portion (e.g., a distal portion) of the configuration of the extraction device 200 depicted in FIG. 7, where the first elongate shaft 202 and the second elongate shaft 214 are depicted in cross-section and the guidewire 111 is depicted from a side view. The configuration of the extraction device 200 depicted in FIGS. 8A-8C includes balloon ports for receiving inflation fluid for adjusting an outer diameter of the first expandable portion 222 and the second expandable portion 224.

As depicted in the FIG. 8A, the guidewire 111 may extend through the second elongate shaft 214 and out of a distal end of the second elongate shaft 214. The first expandable portion 222 may be part of, connected to, and/or otherwise coupled with the first elongate shaft 202 and may be inflatable and/or otherwise expandable by inflation fluid delivered through a balloon port via the first inflation port 230 and the first inflation lumen 231 (not expressly shown in FIG. 8A for clarity purposes). The second expandable portion 224 may be connected to and/or otherwise coupled with the second elongate shaft 214 and may be inflatable and/or otherwise expandable by inflation fluid delivered through a balloon port via the second inflation port 232 and the second inflation lumen 233 (not expressly shown in FIG. 8A for clarity purposes). Although not depicted in FIG. 8A, the first expandable portion 222 and/or the second expandable portion 224 may include one or more support structures (e.g., expandable support structures such as the first expandable support structure 207 and/or the second expandable support structure 209, or other suitable support structure).

FIG. 8B depicts a schematic magnified view of portion 8B from FIG. 8A. The second elongate shaft 214 may include one or more second balloon ports 236 in fluid communication with the second inflation lumen 233 and the second expandable portion located at, along, within, or through any portion of second elongate shaft 214. In operation, fluid may travel into and/or out of the second balloon ports 236 via the second inflation port 232 and the second inflation lumen 233 to expand and contract the second expandable portion 224 and/or the second flexible covering 226.

FIG. 8C depicts a schematic magnified view of portion 8C from FIG. 8A. The first elongate shaft 202 may include one or more first balloon ports 234 located at, along, within, or through any portion of first elongate shaft 202. In operation, fluid may travel into and/or out of the first balloon ports 234 via the first inflation port 230 and the first inflation lumen 231 to expand and contract the first expandable portion 222 and/or the first flexible covering 210.

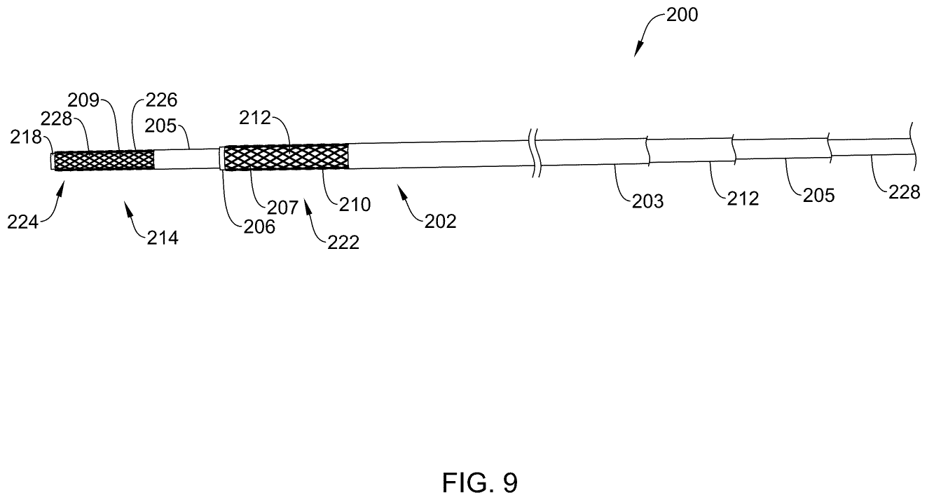

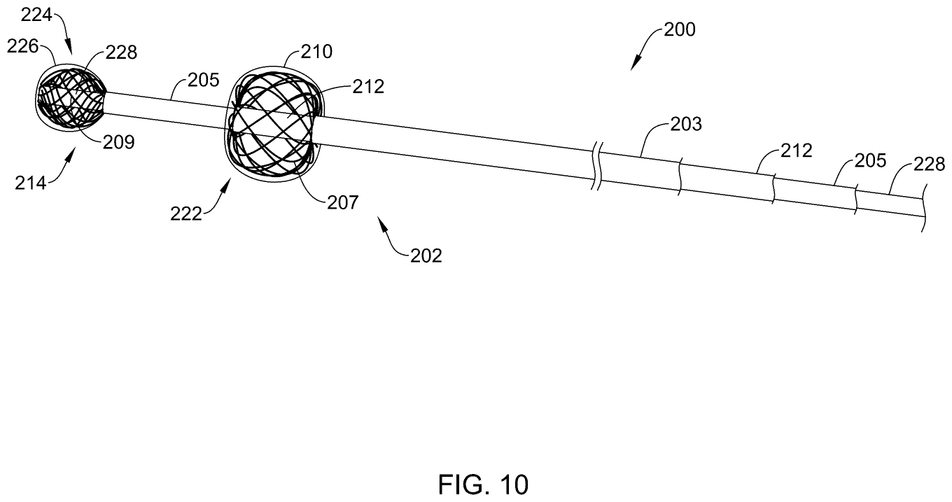

FIGS. 9 and 10 depict schematic views of a portion of an illustrative configuration of the extraction device 200 with the first expandable portion 222 and the second expandable portion 224 configured to adjust (e.g., translate) longitudinally relative to one another and individually adjust between the first position and the second position The first expandable support structure 207 of the first expandable portion 222 and the second expandable support structure 209 of the second expandable portion 224 of the extraction device 200 depicted in FIGS. 9 and 10 may each be and/or may each include a stent structure, however, other suitable configurations are contemplated in which one of the first expandable support structure 207 and the second expandable support structure 209 has a configuration that is different than the stent structure. When the first and/or the second expandable support structure 207, 209 is or includes a stent structure, the respective first and/or second expandable portion 222, 224 may include or may omit the first flexible covering 210 and/or the second flexible covering 226, respectively. In one example, the first expandable support structure 207 may have a stent structure and the first flexible covering 210 and the second expandable support structure 209 may have a stent structure and the second flexible covering 226, as depicted in FIGS. 9 and 10.

At a proximal end of the portion of the extraction device 200 depicted in FIGS. 9 and 10, proximal ends of the tubes of the extraction device 200 have been removed to show an illustrative concentric configuration of those tubes, as described below. Proximal ends of the tubes of the first proximal portion 203 and the second proximal portion 205 may be adjusted relative to one another to longitudinally and/or rotationally translate the first expandable portion 222 and the second expandable portion 224 relative to one another. Proximal ends of the tubes of the first actuator 212 and the second actuator 228 may be adjusted relative to proximal ends of the first proximal portion 203 and the second proximal portion 205, respectively, to radially or laterally adjust the first expandable portion 222 and the second expandable portion 224, respectively.

Any suitable stent structure may be utilized. In some examples, when the first and/or the second expandable support structures 207, 209 are configured with and/or as a stent structure, the stent structure may be configured as a self-expanding stent, a radially biased stent, a foreshortening stent, a non-foreshortening stent, an alloyed stent, a coated or covered stent, a fully coated or covered stent, a partially coated or covered stent, an intermittently coated or covered stents, a synthetic stent, a plastic stent, one or more braided wires, a cut tube, a basket, a filter, and/or other suitable stent structure.

FIG. 9 depicts a schematic side view of a portion of an illustrative configuration of the extraction device 200 in the first position. As shown in FIG. 9, the extraction device 200 may include the first elongate shaft 202 which may include and/or otherwise be coupled with the first proximal portion 203, the first expandable portion 222, the first distal portion 206, the first actuator 212, and/or other suitable components. The first expandable portion 222 may extend from the distal end of the first proximal portion 203 to the proximal end of the first distal portion 206. Although the first coupling component 204 is omitted in the configuration of the extraction device 200 depicted in FIG. 9, other configurations are contemplated that include the first coupling component 204. The first actuator 212, as depicted in FIG. 9 may be an elongate tube with a distal end coupled with the first distal portion 206 and/or a distal end of the first expandable support structure 207 such that when a proximal end of the first actuator 212 is manipulated or actuated by a user, the first expandable portion 222 may adjust between the first position and the second position. Other suitable configurations of the first elongate shaft 202 are contemplated.

As shown in FIG. 9, the extraction device 200 may include the second elongate shaft 214 which may include and/or otherwise be coupled with the second proximal portion 205, the second expandable portion 224, the second distal portion 218, the second actuator 228, and/or other suitable components. The second expandable portion 224 may extend from the distal end of the second proximal portion 205 to the proximal end of the second distal portion 218. Although the second coupling component 216 is omitted in the configuration of the extraction device 200 depicted in FIG. 9, other configurations are contemplated that include the second coupling component 216. The second actuator 228, as depicted in FIG. 9 may be an elongate tube with a distal end coupled with the second distal portion 218 and/or a distal end of the second expandable support structure 209 such that when a proximal end of the second actuator 228 is manipulated or actuated by a user, the first expandable portion 222 may adjust between the first position and the second position. Other suitable configurations of the second elongate shaft 214 are contemplated.

FIG. 10 depicts a schematic perspective view of the portion of the illustrative configuration of the extraction device 200 depicted in FIG. 10 that has been adjusted to the second position. As shown in FIG. 10, both of the first expandable portion 222 and the second expandable portion 224 are in an expanded configuration in response to axial translation or actuation of the first actuator 212 relative to the first proximal portion 203 and the second actuator 228 relative to the second proximal portion 205. The magnitude of expansion of the first and/or second expandable portions 222, 224 may be modified and/or controlled to any desirable and/or feasible magnitude of expansion via the first actuator 212 and/or the second actuator 228.

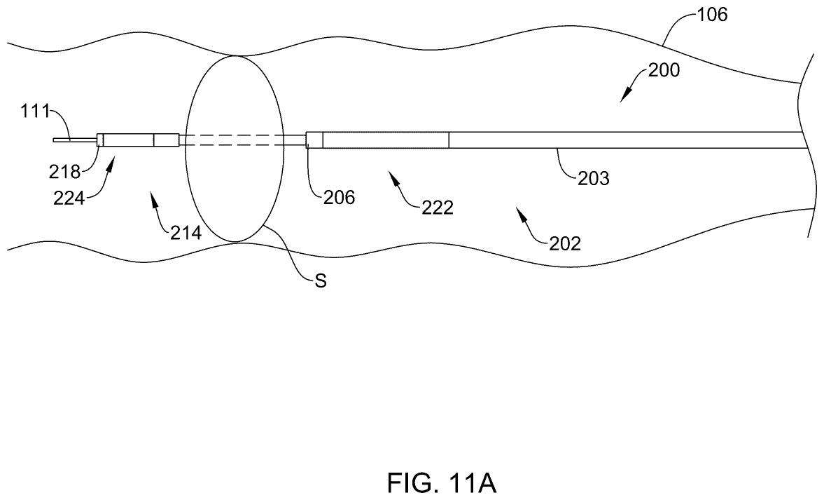

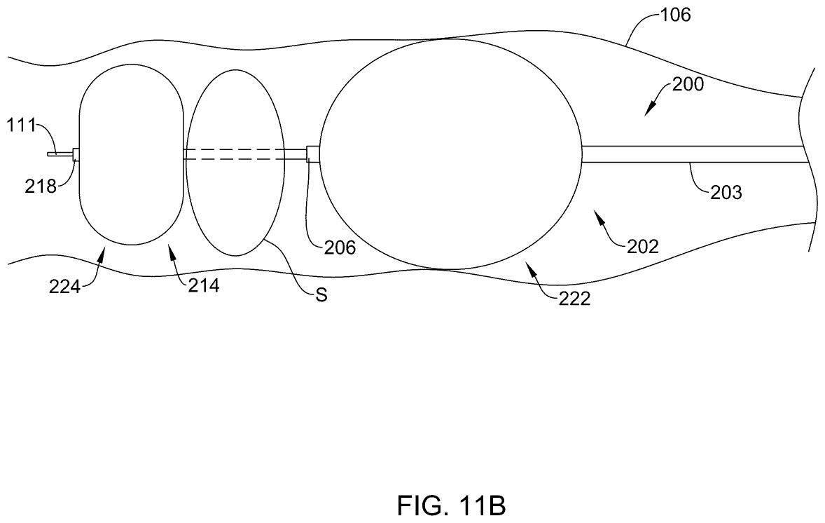

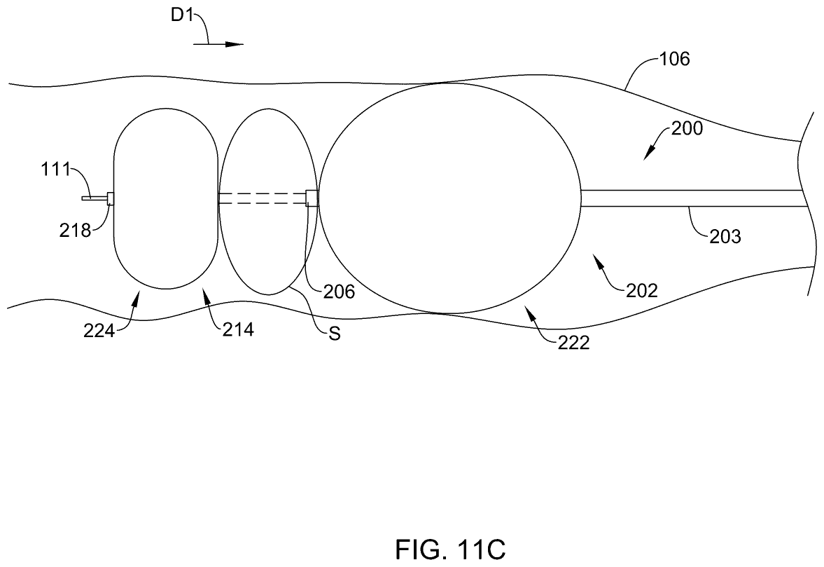

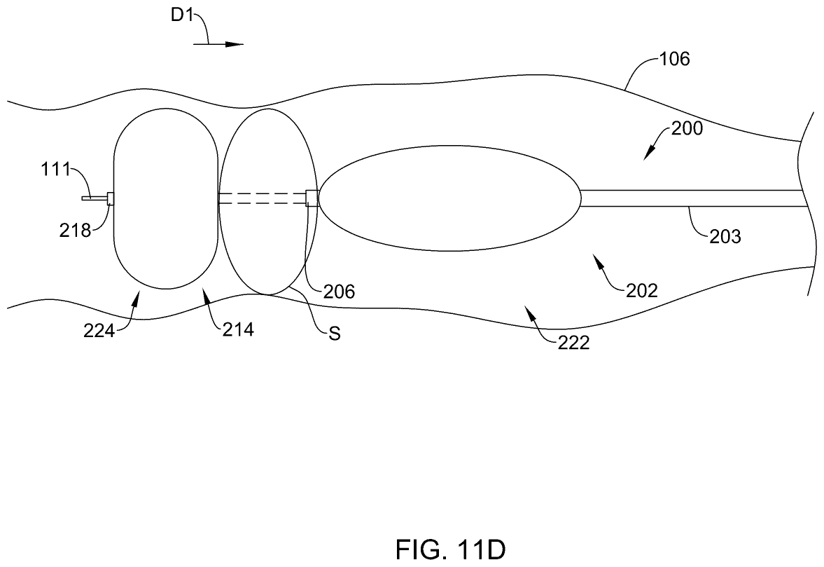

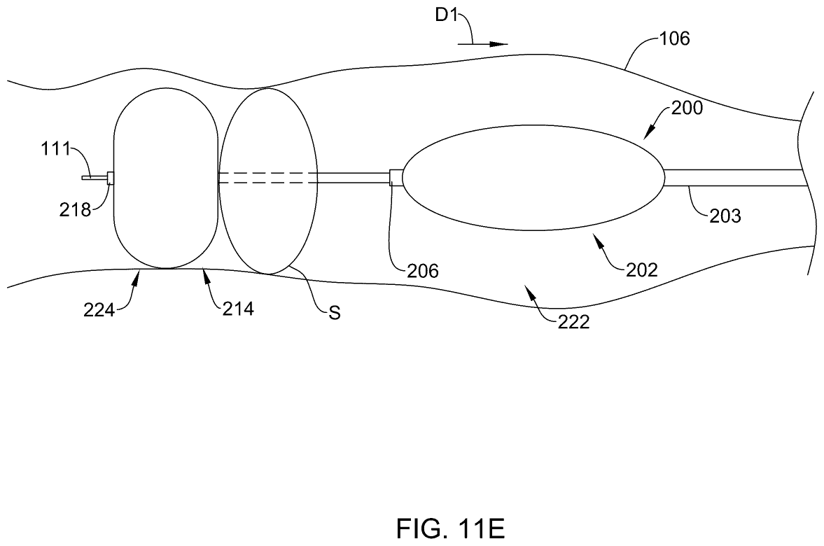

FIG. 11A-E schematically depict views of an illustrative configuration of the extraction device 200 used in a method or technique for removing an anatomical obstruction S (e.g., a stone and/or other suitable anatomical obstruction) from a body lumen of a subject. In this and other examples, the “lumen of a subject” may be a lumen of the biliary duct 106, but the extraction device 200 may be used to remove obstructions in other suitable lumens including, but not be limited to, lumens of a vessel, an organ, a tissue, a concavity, a tract, a sphincter, a valve, and/or any other suitable anatomical structures.

FIG. 11A shows initial positioning of an illustrative configuration of the extraction device 200 deployed at, along, and/or across a desired treatment area of a subject. A desired treatment area of a subject may include, but is not limited to, anywhere along and/or about the biliary tract, anywhere along and/or about the digestive tract, anywhere along and/or about the gastrointestinal tract, anywhere along and/or about the vasculature, anywhere along and/or about the microvasculature, anywhere along and/or about the urinary tract, anywhere along and/or about the cardiovascular system, anywhere along/and or about the circulatory system, anywhere along and/or about the pulmonary system, anywhere along and/or about renal structures, anywhere along and/or about biliary structures, anywhere along and/or about an internal organ, and or any other suitable portion of a body of the subject. In one example and as depicted in FIG. 11A, the extraction device 200 may be inserted into the biliary duct 106, as discussed herein.

As shown in FIG. 11A, the extraction device 200 may be advanced and/or guided in the direction of D2 over the guidewire 111 and inserted into the biliary duct 106 while in the first position (e.g., with the first expandable portion 222 and the second expandable portion 224 in a collapsed configuration) until the extraction device 200 extends across the anatomical obstruction S. The guidewire 111 may be inserted through a dilated access region (e.g., at or proximate the papilla of Vater) proximate the biliary duct 106 (e.g., proximate the biliary tract) prior to insertion of the extraction device 200. The guidewire 111 may be omitted and the extraction device 200 maybe advanced into the biliary duct 106 in one or more other suitable manners.

The extraction device 200 may be positioned proximate the anatomical obstruction S within the lumen of the biliary duct 106. In some examples, the second expandable portion 224 may be advanced to a position distal of the first expandable portion 222, and the extraction device 200 may be located in the biliary duct 106 with the second expandable portion 224 located distal of the anatomical obstruction S and the first expandable portion 222 located proximal of the anatomical obstruction S. When passing the anatomical obstruction S, the extraction device 200 may pass through the anatomical obstruction S and/or around the anatomical obstruction S as space permits.

After positioning the extraction device 200 across the anatomical obstruction S, one or both of the first expandable portion 222 and the second expandable portion 224 may be expanded at the respective locations relative to one another and/or relative to the anatomical obstruction S. In some examples, the first expandable portion 222 may be expanded, as discussed herein or otherwise (e.g., via the first actuator 212, not shown in FIGS. 11A-11E), to dilate the lumen of the biliary duct 106 and/or the surroundings of the anatomical obstruction extraction device 200, as shown in FIG. 11B. In some examples, expanding the first expandable portion 222 may anchor the extraction device 200 at a desired location in the biliary duct 106. The second expandable portion 224 may be expanded, as discussed herein or otherwise (e.g., via the second actuator 228, not shown in FIGS. 11A-11E), to a desirable outer diameter that allows the second expandable portion to adjusted in the direction D1 (e.g., the proximal direction) while mitigating an amount of the anatomical obstruction S or other obstruction (e.g., debris therefrom) that is not withdrawn in the direction D1 with the second expandable portion D1. In some examples, the first expandable portion 222 may be expanded prior to expanding the second expandable portion 224, but other suitable configurations are contemplated in which the first and the second expandable portions 222, 224 are expanded at the same time or the second expandable portion 224 is expanded prior to expanding the first expandable portion 222.

Once the first expandable portion 222 and the second expandable portion 224 are expanded, the second expandable portion 224 may be retracted in the direction D1 (e.g., the proximal direction) and relative to (e.g., toward) the first expandable portion 222, as depicted for example in FIG. 11C. In some examples, the second expandable portion 224 may be withdrawn in the direction D1 relative to the first expandable portion 222 such that the second expandable portion 224 may contact and urge or otherwise move the anatomical obstruction S toward the first expandable portion 222 and toward an opening of the biliary duct 106 (e.g., a the papilla of Vater) and/or out of the duct, lumen, and/or anatomical structure within which it is located. The second expandable portion 224 may be adjusted relative to the first expandable portion 222 by translating the second proximal portion 205 in the direction D1 relative to the first proximal portion 203 and/or in one or more other suitable manners. The anatomical obstruction S may be urged in the opposite direction (e.g., distally) and/or in any direction suitable for efficacy of procedure and safety of the subject by switching the operations of the first expandable portion 222 and the second expandable portion 224.

Once the anatomical obstruction S has been advanced toward the first expandable portion 222, as depicted in FIG. 11C, the first expandable portion 222 may be collapsed (e.g., contracted) to a reduced diameter relative to when the first expandable portion 222 is dilating the lumen of the biliary duct 106, as depicted in FIG. 11D. In some examples, the outer diameter of the first expandable portion 222 may be reduced while the second expandable portion 224 may remain expanded to the desired outer diameter to keep a portion of the lumen of the biliary duct 106 dilated and/or to prevent the anatomical obstruction S from migrating deeper into the lumen. The outer diameter of the first expandable portion 222 may be reduced by releasing the first actuator 212 and/or by further actuating the first actuator 212, but other suitable configurations are contemplated.

Once the first expandable portion 222 has been adjusted to the reduced diameter, the first expandable portion 222 may be translated (e.g., retracted) in the direction D1, as depicted for example in FIG. 11E. The first expandable portion 222 may be adjusted relative to the second expandable portion 224 in the direction D1 by translating the first proximal portion 203 in the direction D1 relative to the second proximal portion 205 and/or in one or more other suitable manners. Once a distance between the first expandable portion 222 and the second expandable portion 224 has increased, as depicted in FIG. 11E relative to a distance therebetween that is depicted in FIG. 11C, the process shown in and described with respect to FIGS. 11B-11E may be repeated, iterated, and/or otherwise repeatedly continued until the anatomical obstruction S is cleared from the lumen of the subject and/or moved to a proper location within the subject, such as a location conducive to disposal of the stone and/or anatomical obstruction (including but not limited to vessels and/or lumens of greater diameter and/or organs that allow for passing of the anatomical obstruction S without or with minimal discomfort)..

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The present disclosure has been described above based on various configurations. Various modifications can be made to the combination of the components and processes in the configurations and that such modifications are included in the scope of the present disclosure.

While configurations of the present disclosure have been described above, it is to be understood that variations and modifications will be apparent to those skilled in the art without departing from the scope and spirit of the disclosure. The scope of the present disclosure, therefore, is to be determined solely by the following claims.

What is claimed is:

1. A medical device comprising:
a first elongate shaft comprising a first expandable portion and a first proximal portion extending in a proximal direction from the first expandable portion, the first expandable portion comprising one or more first elongate support elements;
a first actuator configured to actuate the first expandable portion between a first elongate configuration and a first expanded configuration;
a second elongate shaft concentrically disposed about the first elongate shaft, the second elongate shaft comprising a second expandable portion and a second proximal portion extending in the proximal direction from the second expandable portion, the second expandable portion comprising one or more second elongate support elements; and
a second actuator configured to actuate the second expandable portion between a second elongate configuration and a second expanded configuration.

2. The medical device of claim 1, wherein one or both of the first expandable portion and the second expandable portion include a flexible covering.

3. The medical device of claim 2, wherein the flexible covering is one or more of a balloon, a stent, a sleeve, and a basket.

4. The medical device of claim 2, wherein the flexible covering is substantially porous.

5. The medical device of claim 1, wherein the first expandable portion comprises a balloon covering the one or more first elongate support elements and the first elongate shaft comprises a first inflation port located at the first proximal portion, the first inflation port is configured to be in fluid communication with the balloon and to receive inflation fluid.

6. The medical device of claim 1, wherein the one or more first elongate support elements of the first expandable portion comprise a plurality of circumferentially spaced elongate struts.

7. The medical device of claim 1, wherein one or both of the one or more first elongate support elements of the first expandable portion and the one or more second elongate support elements of the second expandable portion are longitudinally biased toward one of the first elongate configuration and the second elongate configuration.

8. The medical device of claim 1, wherein the first actuator comprises one or more first pull wires coupled with the first expandable portion and the second actuator comprises one or more second pull wires coupled with the second expandable portion.

9. The medical device of claim 1, wherein the first expandable portion is longitudinally adjustable with the first proximal portion when in an expanded configuration and the second expandable portion is longitudinally adjustable with the second proximal portion when in an expanded configuration.

10. The medical device of claim 1, wherein the first elongate shaft comprises a central lumen.

11. An obstruction extraction device comprising:
a first elongate shaft, the first elongate shaft comprising:
a first proximal portion and a first distal portion;
a plurality of first elongate struts extending between the first proximal portion and the first distal portion and circumferentially spaced from one another;
a first flexible covering extending over the plurality of first elongate struts; and
one or more first actuators coupled with the first distal portion, and wherein the plurality of first elongate struts is configured to longitudinally and radially adjust in response to relative longitudinal movement between the first proximal portion and the one or more first actuators; and
a second elongate shaft configured to be concentrically disposed about the first elongate shaft, wherein the first elongate shaft and the second elongate shaft are configured to be longitudinally adjustable with respect to one another, the second elongate shaft comprising:
a second proximal portion and a second distal portion;
a plurality of second elongate struts extending between the second proximal portion and the second distal portion and circumferentially spaced from one another;
a second flexible covering extending over the plurality of second elongate struts; and
one or more second actuators coupled with the second distal portion, wherein the plurality of second elongate struts is configured to longitudinally and radially adjust in response to relative longitudinal movement between the second proximal portion and the one or more second actuators.

12. The obstruction extraction device of claim 11, wherein the plurality of first elongate struts and the plurality of second elongate struts are biased in a longitudinal direction.

13. The obstruction extraction device of claim 11, wherein one or both of the first proximal portion and the second proximal portion include an inflation port.

14. The obstruction extraction device of claim 11, wherein each of the first flexible covering and the second flexible covering is one or more of a balloon, a stent, a sleeve, and a basket.

15. The obstruction extraction device of claim 11, wherein the first flexible covering and the plurality of first elongate struts are longitudinally adjustable with the first proximal portion and the second flexible covering and the plurality of second elongate struts are longitudinally adjustable with the second proximal portion.

16. The obstruction extraction device of claim 11, wherein one or both of the one or more first actuators and the one or more second actuators comprise one or more pull wires.

17. A method of removing an obstruction from a biliary tract of a subject, the method comprising:
inserting a guidewire through a dilated access region proximate the biliary tract;
guiding an obstruction extraction device over the guidewire and positioning the obstruction extraction device proximate the obstruction within the biliary tract;
advancing a first expandable portion of a first elongate shaft to a location in the biliary tract distal of the obstruction, the first expandable portion comprising a first elongate support structure and a first covering;
expanding a second expandable portion of a second elongate shaft at a location in the biliary tract proximal of the obstruction, the second expandable portion comprising a second elongate support structure and a second covering;
expanding the first expandable portion at the location distal of the obstruction; and
retracting the first elongate shaft and the first expandable portion in a proximal direction toward the second expandable portion to advance the obstruction in the proximal direction.

18. The method of claim 17, the method further comprising:
contracting the second expandable portion;
retracting the second expandable portion in the proximal direction after contracting the second expandable portion; and
expanding the second expandable portion after retracting the second expandable portion.

19. The method of claim 17, wherein the first covering of the first expandable portion comprises one or more of a balloon, a stent, a sleeve, and a basket.

20. The method of claim 17, wherein the first elongate support structure of the first expandable portion comprises a plurality of elongate struts circumferentially spaced from one another.
